(19) United States
(12) Patent Application Publication
(10) Pub. No.: US 2014/0275638 A1
(43) Pub. Date: Sep. 18, 2014

(54) METHOD FOR PREPARING TRIOLS AND DIOLS FROM BIOMASS-DERIVED REACTANTS

(71) Applicant: WISCONSIN ALUMNI RESEARCH FOUNDATION, Madison, WI (US)

(72) Inventors: George W. Huber, Middleton, WI (US); Yong Tae Kim, Madison, WI (US)

(73) Assignee: Wisconsin Alumni Research Foundation, Madison, WI (US)

(21) Appl. No.: 13/838,226

(22) Filed: Mar. 15, 2013

(51) Int. Cl. C07C 29/132

(52) U.S. Cl. CPC C07C 29/132
USPC 568/865

(57) ABSTRACT

A method to make triols and diols is described. The method includes the steps of performing an aqueous-phase hydrodeoxygenation reaction on a feedstock containing a biomass-derived reactant in aqueous solution. The feedstock is contacted with a heterogeneous metal-containing bifunctional catalyst or a combination of two or more heterogeneous metal-containing catalysts that catalyze cleavage of C—C and C—O bonds, for a time, temperature, pressure, and weight hourly space velocity to yield a product mix comprising triols, diols, or combinations thereof.

### FIELD OF THE INVENTION

The method described herein is for producing polyols in general, diols in particular, and most specifically α,ω-diols, all from renewable, biomass-derived feedstocks.

### BACKGROUND

Several processes are known for producing diols in general, and α,ω-diols in particular from a petrochemical-derived feedstock. Ethylene glycol, the simplest of the diols, is produced industrially by catalytic hydration of compounds such as ethylene oxide and ethylene carbonate. 1,3-Propanediol is conventionally produced by catalytic hydrogenation of compounds such as 3-hydroxypropanaldehyde. 1,4-Butanediol is produced industrially by catalytic hydrogenation of petroleum-derived compounds such as 1,4-butynediol, 4-hydroxybutylaldehyde, or dimethyl maleate ester. Less commonly used are methods to produce 1,4-butanediol from butadiene, allyl acetate, and succinic acid. 1,5-Pentanediol is produced industrially by catalytic hydrogenation of compounds such as glutaric acid or glutaric esters. 1,5-Pentanediol can also be made from glutaraldehyde. 1,6-Hexanediol is conventionally produced in industrial quantities by catalytic hydrogenation of compounds such as adipic acid or oxocaproic acid. The linear dimerization of acrylic esters also provides an alternative conventional route to 1,6-hexanediol based on a C3 raw material source. Notably, however, the conventional technologies for preparing the required feedstocks required all of the above-noted α,ω-diols requires oxidation of fossil fuel-derived hydrocarbons and subsequent esterification with an alkanol.

Lignocellulosic biomass is not currently commonly used as a feedstock to produce building-block chemicals because current conversion processes are not economically feasible. As long as fossil fuels (crude oil, natural gas, etc.) remain plentiful and can be extracted cheaply, petroleum-derived feedstocks will remain more economically viable that biomass-derived sources. Even faced with that economic reality, there remains a growing market desire to prepare chemicals using more sustainable technologies. Some of this desire is driven by petroleum companies wanting to embrace “socially conscious” or “green” technologies. Some of this desire is driven by the fact that many proven reserves of petroleum are in politically unstable or remote parts of the world (for example, the Middle East, Nigeria, etc.) Whatever the motivation, the conversion of renewable biomass resources into chemicals and fuels traditionally obtained from petroleum is strategically important to improve the sustainability of the chemical industry.

Lignocellulosic biomass is the non-edible portion of biomass. It comprises three major components: cellulose, hemicellulose, and lignin. Extensive research has been carried out regarding the conversion of lignocellulosic biomass into platform molecules. Three main strategies are currently under investigation: (1) Acid-catalyzed dehydration to give furfural from pentoses and hydroxymethylfurfural from hexoses. See, for example, Y. Román-Leshkov, C. J. Barrett, Z. Y. Liu, J. A. Dumesic, Nature 2007, 447, 982-986.). (2) Hydrogenation to give sugar alcohols, most notably sorbitol from glucose. See P. Gallezot, N. Nicolaus, G. Flèche, P. Fuertes, A. Perrard, Journal of Catalysis 1998, 180, 51-55.). (3) Fast pyrolysis to give bio-oils (T. P. Vispute, H. Zhang, A. Sanna, R. Xiao, G. W. Huber, Science 2010, 330, 1222-1227.).

Several recent papers have shown that it is possible to produce α,ω-diols from a 1,2,n-triol precursor (where “n” is an integer designated the position of the third hydroxyl group in the precursor) having three oxygen atoms or cyclic ethers having two oxygen atoms (S. Koso, I. Furikado, A. Shimao, T. Miyazawa, K. Kunimori, K. Tomishige, Chemical Communications 2009, 2035-2037; T. Buntara, S, Noel, P. H. Phua, I. Melián-Cabrera, J. G. de Vries, H. J. Heeres, Angewandte Chemie International Edition 2011, 50, 7083-7087; M. Chia, Y. J. Pagán-Tones, D. Hibbitts, Q. Tan, H. N. Pham, A. K. Datye, M. Neurock, R. J. Davis, J. A. Dumesic, Journal of the American Chemical Society 2011, 133, 12675-12689).

Published International Application WO2011/149339 describes the conversion of 5-hydroxymethyl-2-furfuraldehyde into product compositions containing 2,5-tetrahydrofuran-dimethanol, 1,2,6-hexanetriol, or tetrahydropyran-2-methanol. This reaction is catalyzed using a Raney nickel or nickel catalyst. The products can thereafter be converted into 1,6-hexanediol in the presence of a copper chromite catalyst. The approach described in this publication, however, requires multiple-step reactions performed in a batch reactor using alcohols as a reaction media. The overall reaction proceeds relatively slowly, and thus is not suitable to produce 1,6-hexanediol on a commercial scale.

A recent paper has shown that it is possible to produce 1,4-butanediol from sugar alcohols such as erythritol via a one-pot hydrogenolysis reaction. Running the reaction using an Ir—Re/SiO₂ catalyst gave 1,4-butanediol at a yield of about 33%. (Y. Amada, H. Watanabe, Y. Hirai, Y. Kajikawa, Y. Nakagawa, K. Tomishige, ChemSusChem 2012, 5, 1991-1999). The reaction proceeds relatively slow in a batch reactor.

Aqueous phase processing (APP) (e.g., reforming and hydrodeoxygenation in an aqueous solvent) have the benefit of being continuous, more readily scalable than batch processing, and capable of using a wide variety of reactant feedstocks. Various APP methods and techniques are described in the patent literature. See, for example, U.S. Pat. Nos. 6,699,457; 6,964,757; 6,953,873; 6,964,758; 7,618,612; 7,767,867; and 7,989,664. See also U.S. Patent Publication Nos. 2008/0216391; 2008/0300434; 2008/0300435; 2009/0211942; 2010/0076233; and 2011/0306804. See also PCT Published Application Nos. PCT/US2006/048030 and PCT/US2008/056330. APP methods can be used to generate hydrogen, hydrocarbons, alcohols, and polyols from biomass-derived feedstocks. The yield of any given product depends on a large number of process variables, including the type of feedstock utilized, the space velocity at which the reaction is performed (the mass of reactant per mass of catalyst per unit of time), the reaction temperature, the reaction pressure, solvent concentration, the hydrogen concentration, the reaction byproducts, and the reactivity and robustness of the catalyst used. None of the earlier methods utilizing biomass-derived reactants diols using APP of carbohydrates or lignocellulosic biomass.

U.S. Pat. No. 8,383,866, issued Feb. 26, 2013, describes a process for preparing diols by hydrogenating a mixture comprising carboxylic acids, carboxylic anhydrides and/or carboxylic esters/lactones using a cobalt-containing catalyst. Alkali metal and/or alkaline earth metal ions (excluding alkali metal and/or alkaline earth metal ions of mineral acids) are added to the hydrogenation feed. In this process, the acid number in the feed upstream of the catalyst, which is established by the ratio of feed to circulation over the reactor, is between 10 and 90.

U.S. Pat. No. 8,304,583, issued Nov. 6, 2012, describes a process of hydrogenating an aliphatic dialdehyde, such as a C₆-C₁₆ alicyclic dicarboxaldehyde, to yield an aliphatic diol, preferably, a C₆-C₁₆ alicyclic diol, such as cis/trans-(1,3)(1,4)-cyclohexanedimethanol. The process involves contacting one or more aliphatic dialdehydes in a liquid phase with hydrogen in the presence of a hydrogenation catalyst in a hydrogenation zone and in the presence of water in an amount equal to or greater than 10 wt % percent, based on the weight of the total liquid feed to the hydrogenation zone.

### SUMMARY OF THE INVENTION

Disclosed herein is a method for producing value-added chemicals such as triols, diols, and α,ω-diols from a biomass-derived feedstock. The method utilizes bifunctional catalysts and/or a two-stage catalyst system to drive the hydrodeoxygenation of a liquid (preferably aqueous) solution of carbohydrates to yield triols and diols, including α,ω-diols. The reactions take place in the presence of at least one heterogeneous catalyst. Gaseous H₂ may introduced into the reaction chamber to drive hydrogenation of the incoming feedstock.

Disclosed herein is a method to make triols and diols. The method comprises performing an hydrodeoxygenation reaction on a feedstock. The feedstock itself comprises (i) a biomass-derived reactant in aqueous solution, and hydrogen. The feedstock is contacted with a heterogeneous metal-comprising bifunctional catalyst or a combination of two or more heterogeneous metal-comprising catalysts that catalyze cleavage of C—C and C—O bonds, for a time, temperature, pressure, and weight hourly space velocity to yield a product mix comprising triols, diols, or combinations thereof. In some versions of the method, the feedstock is contacted with the catalyst or catalysts for a time, temperature, pressure, and weight hourly space velocity to yield a product mix comprising α,ω-diols.

The hydrodeoxygenation reaction may optionally be carried out at a temperature from about 100° C. to about 500° C., a pressure from about 100 psig to about 1000 psig, and a weight-hourly space velocity from about 0.1 to about 50.0 gram of liquid feedstock per gram of catalyst per hour. The feedstock may be contacted with the catalyst or catalysts under the conditions stated in the immediately preceding paragraph for a time, temperature, pressure, and weight hourly space velocity to yield a product mix comprising C₂ to C₁₂-α,ω-diols, or C₂ to C₆-α,ω-diols.

In any of the foregoing versions of the method, the catalyst or catalysts may optionally be adhered to a support. The support may comprise a solid acid having Lewis acid functionality or Bronsted acid functionality. The support may be selected from the group consisting of carbon, a refractory oxide, a phosphate-bonded refractory oxide, and combinations thereof.

In some versions of the method, the catalyst or catalysts may comprise a metal selected from the group consisting of Pt, Pd, Ru, Ir, W, Co, Ni, Fe, alloys thereof, mixtures thereof, and combinations thereof. The catalyst or catalysts may further be combined with or alloyed with one or more additional metals selected from the group consisting of alkali metals, alkaline earth metals, and rare earth metals.

A more specific version of the method comprises performing an aqueous-phase hydrodeoxygenation reaction on a feedstock comprising (i) a biomass-derived reactant in aqueous solution, and (ii) hydrogen, as noted earlier. Here, though, the feedstock is contacted with a heterogeneous metal-comprising bifunctional catalyst or a combination of two or more heterogeneous metal-comprising catalysts that catalyze cleavage of C—C and C—O bonds, at a temperature from about 100° C. to about 500° C., a pressure from about 100 psig to about 1000 psig, and a weight-hourly space velocity from about 0.1 to about 50.0 gram of liquid feedstock per gram of catalyst per hour, wherein the catalyst or catalysts comprise a metal selected from the group consisting of Pt, Pd, Ru, Ir, W, Co, Ni, Fe, alloys thereof, mixtures thereof, and combinations thereof, wherein the catalyst or catalysts are adhered to a support selected from the group consisting of carbon, a refractory oxide, a phosphate-bonded refractory oxide, and combinations thereof; and wherein the reaction yields a product mix comprising triols, diols, or combinations thereof. The catalyst or catalysts may further be combined with or alloyed with one or more additional metals selected from the group consisting of alkali metals, alkaline earth metals, and rare earth metals. The feedstock may be contacted with the catalyst or catalysts for a time, temperature, pressure, and weight hourly space velocity to yield a product mix comprising α,ω-diols.

The biomass-derived reactant may be in the form of an aqueous-phase solution of the reactant. The method, however, may also be carried out on solid biomass per se (such as a slurry of sawdust) or a slurry of a biomass-derived reactant.

### BRIEF DESCRIPTION OF THE DRAWINGS

FIG. 1 is a reaction scheme depicting the conversion of complex carbohydrates into sugars and sugar alcohols via hydrolysis and subsequent hydrogenation.

FIG. 2 is a reaction scheme that starts with the sugar alcohols depicted in FIG. 1 and illustrates their conversion to anhydrosorbitols and α,ω-diol precursors such as triols.

FIG. 3 is a scheme that starts with the anhydrosorbitols and triols depicted in FIG. 2 and illustrates their conversion value-added products such as α,ω-diols and branched diols. Taken together, FIGS. 1, 2, and 3 depict an integrated reaction scheme for converting biomass-derived carbohydrates into triols and diols via aqueous-phase hydrodeoxygenation in the presence of a bifunctional heterogeneous catalyst.

FIG. 4 is a schematic diagram of a reactor system that may be used for carrying out the subject method.

FIGS. 5A and 5B are graphs depicting the catalytic performance for the hydrogenation of furfuryl alcohol over 10% Co/γ-Al₂O₃ catalyst, T=413 K, P=2.1 Mpa, WHSV=0.6 h⁻¹, feedstock=2 wt % furfuryl alcohol aqueous solution; H₂ flow rate=40 ml min⁻¹. FIG. 5A depicts conversion percentage (Y-axis) to tetrahydrofurfuryl alcohol (THFA) as a function of time on stream (X-axis). FIG. 5B depicts the carbon selectively percentage (Y-axis) to THFA (solid circles) and ratio of 1,5-propanediol to 1,2-pentanediol (open circles) as function of time on stream (X-axis).

### DETAILED DESCRIPTION

### Abbreviations and Definitions

APHDO=Aqueous-Phase Hydrodeoxygenation.

“Biomass” as used herein includes materials containing cellulose, hemicellulose, lignin, protein and carbohydrates such as starch and sugar. Common forms of biomass include trees, shrubs and grasses, corn and corn husks as well as municipal solid waste, waste paper and yard waste. Biomass high in starch, sugar or protein such as corn, grains, fruits and vegetables, is usually consumed as food. Conversely, biomass high in cellulose, hemicellulose and lignin is not readily digestible by humans and is primarily utilized for wood and paper products, fuel, or is discarded as waste. “Biomass” as used herein explicitly includes branches, bushes, canes, corn and corn husks, energy crops, forests, fruits, flowers, grains, grasses, herbaceous crops, leaves, bark, needles, logs, roots, saplings, short rotation woody crops, shrubs, switch grasses, trees, vegetables, vines, hard and soft woods. In addition, biomass includes organic waste materials generated from agricultural processes including farming and forestry activities, specifically including forestry wood waste. “Biomass” includes virgin biomass and/or non-virgin biomass such as agricultural biomass, commercial organics, construction and demolition debris, municipal solid waste, waste paper, and yard waste. Municipal solid waste generally includes garbage, trash, rubbish, refuse and offal that is normally disposed of by the occupants of residential dwelling units and by business, industrial and commercial establishments, including but not limited to: paper and cardboard, plastics, food scraps, scrap wood, saw dust, and the like.

“Biomass-derived”=Compounds or compositions fabricated or purified from biomass. Glucose for use in the disclosed method may be biomass-derived.

As used herein, the term “bifunctional catalyst” refers without limitation to any catalyst or combination of two or more catalysts, now known or developed in the future, homogenous or heterogeneous, that catalyzes the cleavage of carbon-carbon and carbon-oxygen bonds. Typically such catalysts comprise catalytically active metal sites and catalytically active acid sites, although any catalyst that operates by any underlying mechanism to cleave C—C and C—O bonds may be used in the present method. For example, suitable bifunctional catalysts may comprise a catalytically active metal affixed, bonded, or otherwise adhered to a support. The catalyst may comprise a catalytically active metal selected from the group consisting of Pt, Pd, Ru, Ir, W, Co, Ni, Fe, alloys and mixtures thereof, and combinations of any of the foregoing. Note that the forgoing list recites the preferred catalysts. Other catalytically active metals may also used or included in the bifunctional catalyst. In particular, the activity of the bifunctional catalyst may be enhanced by combining it with, or alloying it with additional metals, such as an alkali metal (e.g., Li, Na, K, Rb, and Cs), an alkaline earth metal (Be, Mg, Ca, Sr, Ba and Ra), and/or a rare earth element (La, Ce, Pr, Nd, Pm, Sm, Eu, Gd, Tb, Dy, Ho, Er, Tm, Yb, Lu, Sc, and Y), or a transition or inner-transition metal (i.e., any element in the d-block of the periodic table, including all of Groups 3 to 12 of the periodic table, and the f-block actinides and lanthanides.) (N.B., the lanthanides are members of the narrower genus of rare earth elements, as well as the broader genus of inner-transition metals.) Preferred transition metals for use in the bifunctional catalyst include V, Cr, Mn, Cu, Zn, and Re).

Brønsted-Lowry Acid/Base=A Brønsted-Lowry acid is defined herein as any chemical species (atom, ion, molecule, compound, complex, etc.), without limitation, that can donate or transfer one or more protons to another chemical species. Mono-protic, diprotic, and triprotic acids are explicitly included within the definition. A Brønsted-Lowry base is defined herein as any chemical species that can accept a proton from another chemical species. Included among Brønsted-Lowry acids are mineral acids, organic acids, heteropolyacids, solid acid catalysts, zeolites, etc. as defined herein. Note that this list is exemplary, not exclusive. The shortened term “Brønsted” is also used synonymously with “Brønsted-Lowry.”

“Carbohydrate” is defined herein as a compound that consists only of carbon, hydrogen, and oxygen atoms, in any ratio.

“C₅ carbohydrate” refers to any carbohydrate, without limitation, that has five (5) carbon atoms. The definition includes pentose sugars of any description and stereoisomerism (e.g., D/L aldopentoses and D/L ketopentoses). C₅ carbohydrates include (by way of example and not limitation) arabinose, lyxose, ribose, ribulose, xylose, and xylulose.

“C₆ carbohydrate” refers to any carbohydrate, without limitation, that has six (6) carbon atoms. The definition includes hexose sugars of any description and stereoisomerism (e.g., D/L aldohexoses and D/L ketohexoses). C₆ carbohydrates include (by way of example and not limitation) allose, altrose, fructose, galactose, glucose, gulose, idose, mannose, psicose, sorbose, tagatose, and talose.

“Cellulose” refers to a polysaccharide of glucose monomers ((C₆H₁₀O₅)ₙ); “cellulosic biomass” refers to biomass as described earlier that comprises cellulose, and/or consists essentially of cellulose, and/or consists entirely of cellulose. Lignocellulosic biomass refers to biomass comprising cellulose, hemicellulose, and lignin. Lignocellulosic biomass comprises xylose, as does hemicellulose. For the experiments described below, microcrystalline cellulose (5% moisture, average size 20 μm) was obtained from Sigma-Aldrich, St. Louis, Mo. Dried corn stover was obtained through the Great Lakes Bioenergy Research Center, Madison, Wis., USA.

“Dehydration catalyst” means any catalyst, without limitation, whether now known or developed in the future, capable of removing water from organic compounds.

“Glucose-containing oligomers, glucose-containing polymers, Glucose-containing reactant, C6-containing reactant”=Any chemical species, having any type of intramolecular bond type, that comprises a glucose unit. The definition explicitly includes glucose-containing disaccharides (such as, but not limited to, sucrose, lactose, maltose, trehalose, cellobiose, kojibiose, nigerose, isomaltose, β,β-trehalose, α,β-trehalose, sophorose, laminaribiose, gentiobiose, turanose, maltulose, palatinose, gentiobiulose, etc.), trisaccharides (such as, but not limited to, isomaltotriose, nigerotriose, maltotriose, maltotriulose, raffinose, etc.), and larger oligosaccharides and polysaccharides, as well as large and more complex glucose-containing polymers and carbohydrates, such as, but not limited to, starch, amylase, amylopectin, glycogen, cellulose, hemicelluloses (e.g., xyloglucan, glucomannan, etc.), lignocellulose, and the like. Linear, branched, and macrocyclic oligomers and polymers containing glucose are explicitly included within the definition.

“Heteropolyacid”=A class of solid-phase acids exemplified by such species as H₄SiW₁₂O₄₀, H₃PW₁₂O₄₀, H₆P₂W₁₈O₆₂, H₃₊ₓPMo₁₂₋ₓVₓO₄₀ and the like.

Heteropolyacids may be used as acid catalysts in the present method. Heteropolyacids are solid-phase acids having a well-defined local structure, the most common of which is the tungsten-based Keggin structure. The Keggin unit comprises a central PO₄ tetrahedron, surrounded by 12 WO₆ octahedra. The standard unit has a net (⁻3) charge, and thus requires three cations to satisfy electroneutrality. If the cations are protons, the material functions as a Brønsted acid. The acidity of these compounds (as well as other physical characteristics) can be “tuned” by substituting different metals in place of tungsten in the Keggin structure. See, for example, Bardin et al. (1998) “Acidity of Keggin-Type Heteropolycompounds Evaluated by Catalytic Probe Reactions, Sorption Microcalorimetry and Density Functional Quantum Chemical Calculations,” J. of Physical Chemistry B, 102:10817-10825.

“Homogeneous catalyst”=A catalyst that exists in the same phase (solid, liquid, or gas) as the reactants under reaction conditions. “Heterogeneous catalyst”=A catalyst that exists in a different phase than the reactants under reaction conditions.

“Hydrofuran” is used herein to refer to any unsubstituted or substituted cyclic ester having a single oxygen heteroatom in the ring, and having five total atoms in the ring and which is derived from furanic compounds. Hydrofurans that are miscible in water, such as tetrahydrofuran (THF), are more appropriate for use in the monophasic reactions described herein. In the biphasic reactions, any hydrofuran may be used.

“Hydropyran” is used herein to refer to any unsubstituted or substituted cyclic ester having a single oxygen heteroatom in the ring, and having six total atoms in the ring and which is derived from pyranic compounds. Hydropyrans miscible in water are more appropriate for use in the monophasic reactions described herein. In the biphasic reactions, any hydropyran may be used.

Mineral acid=any mineral-containing acid, including (by way of example and not limitation), hydrochloric acid, nitric acid, phosphoric acid, SA, boric acid, hydrofluoric acid, hydrobromic acid, and the like. Organic acid=any organic acid, without limitation, such as toluenesulfonic acid, formic acid, acetic acid, trifluoroacetic acid, oxalic acid, and the like.

Lewis Acid/Base=A Lewis acid is defined herein as any chemical species that is an electron-pair acceptor, i.e., any chemical species that is capable of receiving an electron pair, without limitation. A Lewis base is defined herein as any chemical species that is an electron-pair donor, that is, any chemical species that is capable of donating an electron pair, without limitation.

The Lewis acid (also referred to as the Lewis acid catalyst) may be any Lewis acid based on transition metals, lathanoid metals, and metals from Group 4, 5, 13, 14 and 15 of the periodic table of the elements, including boron, aluminum, gallium, indium, titanium, zirconium, tin, vanadium, arsenic, antimony, bismuth, lanthanum, dysprosium, and ytterbium. One skilled in the art will recognize that some elements are better suited in the practice of the method. Illustrative examples include AlCl₃, (alkyl)AlCl₂, (C₂H₅)₂AlCl, (C₂H₅)₃Al₂Cl₃, BF₃, SnCl₄ and TiCl₄.

The Group 4, 5 and 14 Lewis acids generally are designated by the formula MX₄; wherein M is Group 4, 5, or 14 metal, and X is a halogen independently selected from the group consisting of fluorine, chlorine, bromine, and iodine, preferably chlorine. X may also be a psuedohalogen. Non-limiting examples include titanium tetrachloride, titanium tetrabromide, vanadium tetrachloride, tin tetrachloride and zirconium tetrachloride. The Group 4, 5, or 14 Lewis acids may also contain more than one type of halogen. Non-limiting examples include titanium bromide trichloride, titanium dibromide dichloride, vanadium bromide trichloride, and tin chloride trifluoride.

Group 4, 5 and 14 Lewis acids useful in the method may also have the general formula MRₙX₄₋ₙ; wherein M is Group 4, 5, or 14 metal; wherein R is a monovalent hydrocarbon radical selected from the group consisting of C₁ to C₁₂ alkyl, aryl, arylalkyl, alkylaryl and cycloalkyl radicals; wherein n is an integer from 0 to 4; and wherein X is a halogen independently selected from the group consisting of fluorine, chlorine, bromine, and iodine, preferably chlorine. X may also be a psuedohalogen. Non-limiting examples include benzyltitanium trichloride, dibenzyltitanium dichloride, benzylzirconium trichloride, dibenzylzirconium dibromide, methyltitanium trichloride, dimethyltitanium difluoride, dimethyltin dichloride and phenylvanadium trichloride.

Group 4, 5 and 14 Lewis acids useful in method may also have the general formula M(RO)ₙR′ₘX₍ₘ₊ₙ₎; wherein M is Group 4, 5, or 14 metal; RO is a monovalent hydrocarboxy radical selected from the group consisting of C₁ to C₃₀ alkoxy, aryloxy, arylalkoxy, alkylaryloxy radicals; R′ is a monovalent hydrocarbon radical selected from the group consisting of C₁ to C₁₂ alkyl, aryl, arylalkyl, alkylaryl and cycloalkyl radicals; n is an integer from 0 to 4; m is an integer from 0 to 4 such that the sum of n and m is not more than 4; and X is a halogen independently selected from the group consisting of fluorine, chlorine, bromine, and iodine, preferably chlorine. X may also be a psuedohalogen. Non-limiting examples include methoxytitanium trichloride, n-butoxytitanium trichloride, di(isopropoxy)titanium dichloride, phenoxytitanium tribromide, phenylmethoxyzirconium trifluoride, methyl methoxytitanium dichloride, methyl methoxytin dichloride and benzyl isopropoxyvanadium dichloride.

Group 5 Lewis acids may also have the general formula MOX₃; wherein M is a Group 5 metal; X is a halogen independently selected from the group consisting of fluorine, chlorine, bromine, and iodine, preferably chlorine. A non-limiting example is vanadium oxytrichloride.

The Group 13 Lewis acids have the general formula MX₃; wherein M is a Group 13 metal and X is a halogen independently selected from the group consisting of fluorine, chlorine, bromine, and iodine, preferably chlorine. X may also be a psuedohalogen. Non-limiting examples include aluminum trichloride, boron trifluoride, gallium trichloride, indium trifluoride, and the like.

The Group 13 Lewis acids useful in method may also have the general formula: MRₙX₃₋ₙ, wherein M is a Group 13 metal; R is a monovalent hydrocarbon radical selected from the group consisting of C₁ to C₁₂ alkyl, aryl, arylalkyl, alkylaryl and cycloalkyl radicals; and n is an number from 0 to 3; and X is a halogen independently selected from the group consisting of fluorine, chlorine, bromine, and iodine, preferably chlorine. X may also be a psuedohalogen. Non-limiting examples include ethylaluminum dichloride, methylaluminum dichloride, benzylaluminum dichloride, isobutylgallium dichloride, diethylaluminum chloride, dimethylaluminum chloride, ethylaluminum sesquichloride, methylaluminum sesquichloride, trimethylaluminum and triethylaluminum.

Group 13 Lewis acids useful in this disclosure may also have the general formula M(RO)ₙR′ₘX₃₋₍ₘ₊ₙ₎; wherein M is a Group 13 metal; RO is a monovalent hydrocarboxy radical selected from the group consisting of C₁ to C₃₀ alkoxy, aryloxy, arylalkoxy, alkylaryloxy radicals; R′ is a monovalent hydrocarbon radical selected from the group consisting of C₁ to C₁₂ alkyl, aryl, arylalkyl, alkylaryl and cycloalkyl radicals; n is a number from 0 to 3; m is an number from 0 to 3 such that the sum of n and m is not more than 3; and X is a halogen independently selected from the group consisting of fluorine, chlorine, bromine, and iodine, preferably chlorine. X may also be a psuedohalogen. Non-limiting examples include methoxyaluminum dichloride, ethoxyaluminum dichloride, 2,6-di-tert-butylphenoxyaluminum dichloride, methoxy methylaluminum chloride, 2,6-di-tert-butylphenoxy methylaluminum chloride, isopropoxygallium dichloride and phenoxy methylindium fluoride.

Group 13 Lewis acids useful in this disclosure may also have the general formula M(RC(O)O)ₙR′ₘX₃₋₍ₘ₊ₙ₎; wherein M is a Group 13 metal; RC(O)O is a monovalent hydrocarbacyl radical selected from the group consisting of C₂ to C₃₀ alkacyloxy, arylacyloxy, arylalkylacyloxy, alkylarylacyloxy radicals; R′ is a monovalent hydrocarbon radical selected from the group consisting of C₁ to C₁₂ alkyl, aryl, arylalkyl, alkylaryl and cycloalkyl radicals; n is a number from 0 to 3 and m is a number from 0 to 3 such that the sum of n and m is not more than 3; and X is a halogen independently selected from the group consisting of fluorine, chlorine, bromine, and iodine, preferably chlorine. X may also be a psuedohalogen. Non-limiting examples include acetoxyaluminum dichloride, benzoyloxyaluminum dibromide, benzoyloxygallium difluoride, methyl acetoxyaluminum chloride, and isopropoyloxyindium trichloride.

The most preferred Lewis acids for use in the method are metal halides generally and more specifically transition metal halides, lathanoid metal halides, and Group 5, 13, and 14 metal halides. Preferred among the metal halides are metal chlorides. Preferred transition metal chlorides include, but are not limited to, TiCl₄, VCl₃. and the like. Preferred Group 13 and 14 metal halides and chlorides include, but are not limited to, BF₃, AlCl₃, SnCl₄, InCl₃, and GaCl₃. Preferred lanthanoid chlorides include, but are not limited to, LaCl₃, DyCl₃ and YbCl₃.

The terms “solid acid” and “solid acid catalyst” are used synonymously herein and can comprise one or more solid acid materials. The solid acid catalyst can be used independently or alternatively can be utilized in combination with one or more mineral acid or other types of catalysts. Solid acid catalysts may be used as catalyst supports in the present method. Exemplary solid acid catalysts which can be utilized include, but are not limited to, heteropolyacids, acid resin-type catalysts, mesoporous silicas, acid clays, sulfated zirconia, molecular sieve materials, zeolites, and acidic material on a thermo-stable support. Where an acidic material is provided on a thermo-stable support, the thermo-stable support can include for example, one or more of silica, tin oxide, niobia, zirconia, titania, carbon, alpha-alumina, and the like. The oxides themselves (e.g., ZrO₂, SnO₂, TiO₂, etc.) which may optionally be doped with additional acid groups such as SO₄²⁻ or SO₃H may also be used as solid acid catalysts.

Further examples of solid acid catalysts include strongly acidic ion exchangers such as cross-linked polystyrene containing sulfonic acid groups. For example, the Amberlyst®-brand resins are functionalized styrene-divinylbenzene copolymers with different surface properties and porosities. (These types of resins are designated herein as “Amb” resins, followed by a numeric identifier of the specific sub-type of resin where appropriate.) The functional group is generally of the sulfonic acid type. The Amberlyst®-brand resins are supplied as gellular or macro-reticular spherical beads. (Amberlyst® is a registered trademark of the Dow Chemical Co.) Similarly, Nafion®-brand resins are sulfonated tetrafluoroethylene-based fluoropolymer-copolymers which are solid acid catalysts. Nafion® is a registered trademark of E. I. du Pont de Nemours & Co.)

Solid catalysts can be in any shape or form now known or developed in the future, such as, but not limited to, granules, powder, beads, pills, pellets, flakes, cylinders, spheres, or other shapes.

Zeolites may also be used as solid acid catalysts and as catalyst supports in the present method. Of these, H-type zeolites are generally preferred, for example zeolites in the mordenite group or fine-pored zeolites such as zeolites X, Y and L, e.g., mordenite, erionite, chabazite, or faujasite. Also suitable are ultrastable zeolites in the faujasite group which have been dealuminated.

The catalysts described herein may be disposed on a catalyst support material, such as the solid acids listed previously, carbon, a refractory oxide, and/or a phosphate-bonded refractory oxide. For example, exemplary refractory oxides include all forms of alumina, zirconia, titania, hafnia, silica, phosphate-bonded versions thereof, and mixtures thereof. (The forgoing list is not exhaustive.) Methods to make phosphate-bonded refractory oxides are described in, for example, U.S. Pat. Nos. 6,258,742 and 6,740,299, which are incorporated herein by reference. The catalyst support material can be or can comprise rare earth-modified refractory metal oxides, where the rare earth may be any rare earth metal, for example, lanthanum or yttrium; and/or alkali earth metal-modified refractory oxides. The catalyst support material can be categorized as materials having a substantially stable surface area at reaction conditions, for example, a surface area that is not substantially altered by reaction conditions or altered in a way that affects the reaction. Supports with relatively high surface areas are generally preferred to ensure that the reactions proceed as quickly and efficiently as possible.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All references to singular characteristics or limitations shall include the corresponding plural characteristic or limitation, and vice-versa, unless otherwise specified or clearly implied to the contrary by the context in which the reference is made.

The processes described herein can be run in batch mode, semi-continuous mode, and/or continuous mode, all of which are explicitly included herein.

All combinations of method or process steps as used herein can be performed in any order, unless otherwise specified or clearly implied to the contrary by the context in which the referenced combination is made.

The methods described and claimed herein can comprise, consist of, or consist essentially of the essential elements and limitations of the disclosed methods, as well as any additional or optional ingredients, components, or limitations described herein or otherwise useful in synthetic organic chemistry.

### Overview:

Referring to FIGS. 1, 2, and 3 together, these figures present an integrated reaction scheme for converting biomass-derived carbohydrates into value-added triols and diols via aqueous-phase hydrodeoxygenation (APHDO) in the presence of a bifunctional heterogeneous catalyst. In the past, APHDO has focused on converting biomass-derived carbohydrates to alkanes for use as liquid transportation fuels. However, diols in general, and α,ω-diols in particular are value-added chemicals that are extensively used as industrial building blocks to make, for example, polyester fibers and films, such as poly(ethylene terephtahalate) (PET), poly(trimethylene terephthalate) (PTT), and poly(butylenes phthalate). Diols are also used in the manufacture of a host of polyurethanes and other polymers, such as poly(tetramethylene ether glycol) (PTMEG, also known as polytetrahydrofuran). These diols are conventionally fabricated from petroleum.

FIG. 1 is a reaction scheme showing that complex carbohydrates derived from any type of biomass (see definition above) can be converted into sugars and sugar alcohols via hydrolysis and subsequent hydrogenation. These sugars and sugar alcohols typically have very good solubility in water. Thus, they are ideal feedstock chemicals for APHDO reactions. FIG. 2 starts where the reactions depicted in FIG. 1 end. Thus, the reaction scheme shown in FIG. 2 starts with the sugar alcohols depicted in FIG. 1 and illustrates their conversion to anhydrosorbitols and α,ω-diol precursors such as 1,2,n-triols. The triols depicted in FIG. 2 (which are also referred to herein as α,ω-diol precursors) are themselves value-added chemicals. Thus, the triols are considered both final products, as well as precursors or intermediates in the production of α,ω-diols. FIG. 3 is a final reaction scheme that starts with the anhydrosorbitols and 1,2,n-triols depicted in FIG. 2 and illustrates their conversion value-added products such as α,ω-diols and branched diols. Specifically, the anhydrosorbitol and 1,2,n-triol reactants can be hydrolyzed to yield tetrahydropyrans and tetrahydrofurans, which are the hydrodeoxygenated to yield linear diols (including α,ω-diols) and branched diols. As shown in FIG. 3, additional hydrogenation will convert the diols into mono-hydric alcohols, as shown in the lower right of FIG. 3.

An exemplary reaction schematic to carry out the method disclosed herein is shown in FIG. 4. here, the carbohydrate feedstock 12 (preferably an aqueous solution of from 2 wt % to 98 wt % carbohydrate) is fed into dehydration/hydrogenation reactor 14, along with hydrogen 10. The intermediates form the dehydration/hydrogenation reactor 14 are fed into hydrogenolysis reactor 16 to yield a final product mix. The product mix is then fed to separator 18. The separator can be any device now known or developed in the future for separating gaseous versus liquid products, as well as organic versus aqueous products, etc. A portion of (or all of) the hydrogen found in the product mix may be recycled via conduit 22 for injection back into dehydration reactor 14. Similarly, a liquid recycle conduit 24 may also be provided to recycle unreacted or partially reacted compounds (e.g., sugar alcohols, 1,2,n-triols) for another pass through the reactor. Waste water is tapped off at 30 for treatment and ultimately discharged back to the environment. an at conduit 28. The product mix exiting separator 20 comprises an organic product stream 26 comprising products with little or no solubility in water (e.g., monohydric alcohols such as hexanols) and an aqueous product stream 28 comprising water-soluble products such as diols. Note that in FIG. 4, reactors 14 and 16 are depicted as physically separate reactors. This is one acceptable configuration. However, reactors 14 and 16 may also be integrated into a single reactor having one or more catalyst beds, as indicated by dashed line 32 in FIG. 4.

It is preferred, although not required, that the amount of hydrogen introduced into the reactor in excess of the theoretical stoichiometric amount required to convert fully the reactant to a diol product. Typically, hydrogen is present in at least 10× excess, and may be present in as much as 1000× excess.

Additionally, it has been found that the yield of diols in general and α,ω-diols in particular is maximized when catalytically active metal sites are in extremely close physical proximity to catalytically active acid sites. It is preferred that these two types of catalytically active sites be brought into intimate association so that the different catalytic sites are physically juxtaposed on an atomic distance scale—typically with average distances between the two types of sites (for example a metal vs a metal oxide) being on the order of no more than about 10 nm.

In this fashion, biomass-derived carbohydrates can be converted into value-added diols and triols.

### Methodology:

The method comprises reacting an aqueous solution comprising at least one biomass-derived carbohydrate reactant, in the presence of a heterogeneous bifunctional catalyst or a collection of heterogeneous catalysts capable of catalyzing the aqueous phase hydrodeoxygenation of the carbohydrate reactant to yield a product mixture comprising triols and diols in general and α,ω-diols in particular.

The reactions are conducted under conditions where the residence time of either the liquid or gas over the catalyst is appropriate to generate α,ω-diols and α,ω-diol precursors, typically cyclic ethers or triols. For aqueous solutions of carbohydrates, the carbohydrates are typically in the condensed liquid phase and the injected hydrogen remains in the gas phase. For example, the weight hourly space velocity (WHSV) for the reaction may be from about 0.1 to about 50.0 gram of liquid feedstock per gram of catalyst per hour (i.e., about 0.1 to about 50 hr⁻¹). The biomass-derived reactant preferably comprises one or more C₄ to C₆ sugar alcohols. The concentration of the biomass-derived reactant in the feedstock solution may be from about 2 wt % to about 98 wt %, based on solvent weight (water). Hydrogen may be fed in the range of from about 0.1 to about 100 mole of hydrogen atom per mole of carbon in liquid feedstock. The hydrogen may be generated in situ using aqueous phase reforming, or external H₂ may be supplied, or H₂ generated downstream may be recycled back into the reaction. The method may be performed in batch fashion or continuously.

The reaction temperatures and pressures may be selected to more favorably produce the desired products in a mixed phase having both a liquid and vapor phase. The reactions may be carried out at temperatures from about 100° C. to about 500° C., or about 100° C. to about 300° C., or about 100° C. to about 250° C. The reaction may be carried out at pressures from 100 psig to 1000 psig or 200 to 900 psig.

The bifunctional catalyst systems used in the method typically involve catalytically active metal and catalytically active acid sites. The acid sites may be Bronsted acids and/or Lewis acids. (See the definitions section.) The ratio of C—O vs C—C bond cleavage can be tuned by adjusting the concentration of metal to acid sites as well as changing the type of metal catalytic sites. The supported catalyst may be used in which the total content of active metal is from about 0.5 to about 20 wt %, although concentrations above and below this range are explicitly within the scope of the attached claims. See the definitions above for the various metal catalysts and catalyst supports that may be used in the method.

### Examples

The following examples are included solely to provide a more thorough description of the method disclosed and claimed herein. The examples are not intended to limit the scope of the claims in any fashion.

### Catalyst Preparation:

High surface area zirconium phosphate (S_{BET}=361 m² g⁻¹, total pore volume=1.05 cm³ g⁻¹) was prepared by co-precipitation using 1.0 mol L⁻¹ ZrCl₂O.8H₂O (Aldrich, St. Louis, Mo.) and 1.0 mol L⁻¹ NH₄H₂PO₄ (Aldrich) aqueous solutions at a molar ratio of P/Zr=2, as described by Y. Kamiya, S. Sakata, Y. Yoshinaga, R. Ohnishi, T. Okuhara, Catal. Lett. 94 (2004) 45-47. The precipitate was aged at room temperature for 30 min, filtered, washed with deionized water until the pH was 5, dried at 373 K overnight, and then calcined in air at 673 K for 4 h. Activated carbon (Norit Darco, 12×40, S_{BET}=650 m² g⁻¹, total pore volume=0.95 cm³ g⁻¹), supplied by Norit Americas, Inc. (Marshall, Tex.), was used as a support. Pt/Zr—P and Pt/C catalysts were prepared by incipient wetness impregnation with an aqueous solution of Pt(NH₃)₄(NO₃)₂ (Strem Chemicals, Newburyport, Mass.). The content of Pt was 4 wt % for Pt/Zr—P. The Pt/Zr—P catalyst was calcined at 533 K for 3 h in air and reduced at 723 K for 2 h in hydrogen before a reaction. Pt—ReOₓ supported on activated carbon (Pt—ReOₓ/C) was prepared by sequentially impregnating Pt/C dried at 373 K with an aqueous solution of HReO₄ (Aldrich). The content of Pt was 5 wt % and that of Re was 4.9 wt % (Re/Pt=1) for Pt—ReOₓ/C. The Pt—ReOₓ catalyst was reduced at 723 K for 2 h in hydrogen before reaction.

### Catalyst Characterization:

Brunauer-Emmett-Teller (BET) surface areas were calculated from nitrogen adsorption data at 77 K obtained using a Quantachrome Autosorb® iQ2 automated gas sorption system (Quantachrome Instruments, Boynton Beach, Fla.). Before the measurements, the sample was degassed under vacuum at 523 K for 12 h. The pore volume and average pore diameter for each catalyst were calculated by the Barrett-Joyner-Halenda (BJH) desorption method. (See table 4 in the examples.)

Static hydrogen chemisorption was carried out at 298 K using a Quantachrome Autosorb® iQ2 automated gas sorption system; each sample (300 mg) was reduced in situ at 723 K for 2 h at a heating rating of 1 K min⁻¹ in flowing hydrogen (50 mL min⁻¹) prior to chemisorption measurements. After the reduction was complete, the sample was degassed at 723 K for 2 h in He. The sample cell was cooled to 298 K followed by evacuation under vacuum for 2 h. The hydrogen monolayer uptake was calculated by subtracting the reversible adsorption from the total hydrogen adsorption.

Temperature-programmed desorption of ammonia (NH₃-TPD) was conducted over 300 mg of each sample in the temperature range 373-973 K at a heating rate of 10 K min⁻¹ under a constant He flow at 12 mL min⁻¹ while monitoring thermal conductivity detector signals (Quantachrome ChemBET Pulsar™ TPR/TPD Automatic Chemisorption Analyzer), after sample saturation with NH₃ at 373 K for 30 min and He flushing for 2 h. The sample was held at 973 K for an additional 2 h. All of the samples were treated at 673 K for 1.5 h in He before the experiments to remove adsorbed water or organic species. Pt—ReOₓ/C was reduced at 723 K for 2 h in hydrogen and passivated at 298 K with 2 mol % O₂ in He before the He pretreatment. The oxygen passivation results in near-complete reoxidation of surface Re species. The peak area can be correlated with the amount of adsorbed NH₃ on the basis of pulsed NH₃ injection experiments.

Temperature-programmed desorption of isopropylamine (IPA-TPD) was conducted over 50-60 mg of each sample in the temperature range 318-973 K at a heating rate of 10 K min⁻¹ under a constant He flow at 100 mL min⁻¹ while monitoring the derivative weight loss and heat flow using thermogravimetric analysis-mass spectrometry (TGA-MS; TA Instruments SDT Q600 system) (TA Instruments, Inc. New Castle, Del.) after sample saturation with IPA at 318 K for 20 min and He flushing for 2 h. All of the samples were treated at 673 K for 1.5 h in He before the experiments. Pt—ReOₓ/C was reduced at 723 K for 2 h in hydrogen and passivated at 298 K with 2 mol % O₂ in He before the He pretreatment. The number of Brønsted acid sites was calculated from the weight loss of the sample where IPA decomposed to form NH₃ and propylene. The amount of propylene was monitored using the mass signal.

Temperature-programmed oxidation was conducted with 50 mg of each sample in the temperature range 318-973 K at a heating rate of 10 K min⁻¹ under a constant air flow at 100 mL min⁻¹ while monitoring the derivative weight loss and heat flow using TGA-MS (TA instruments SDT Q600 system). The sample was purged with air at 318 K for 1 h prior to the temperature programmed oxidation experiments.

The bulk crystalline structure of the catalysts was determined by X-ray diffraction (XRD). The XRD patterns were obtained with a Scintag Pad V using Cu Kα radiation (λ=0.15406 nm), operated at 45 kV and 40 mA (2.0 kW) at a scan rate of 0.1° (2θ) s⁻¹. (Scintag Inc, Cupertino, Calif.) Each sample was reduced at 723 K for 2 h in hydrogen and passivated prior to XRD experiments. The assignment of the crystalline phases was carried out using JADE 9.0 software package (Rigaku Americas, Inc., The Woodlands, Tex.) for the ICDD database.

The amount of carbon formed on the used catalysts was determined using a total organic carbon (TOC) analyzer (Shimadzu TOC-5000A) Shimadzu America, Inc., Columbia, Md.) after the samples were pretreated with He at 518 K to remove weakly chemisorbed oxygenated hydrocarbons before the carbon analysis. During measurement, the temperature was held at 1173 K in flowing oxygen. This combustion process was repeated several times until the amount of carbon became zero. The surface carbon content was calculated by subtracting the amount of carbon on the fresh catalyst from that on the used catalyst.

### Catalytic Performance Studies:

APHDO reactions were conducted in a stainless-steel tubular flow reactor, with both liquid and hydrogen gas, moving in an upflow direction, heated by a Thermcraft Inc. (No. 114-12-1ZH) furnace (Thermacraft, Inc., Winston-Salem, N.C.). The reactor was 30 cm long and had an outer diameter of 6.35 mm. A uniform temperature profile along the catalyst bed was achieved using aluminum filler inserted in the void between the furnace and the tubular reactor. For catalytic activity measurements, 3.3 g of the catalyst without diluents were loaded into the reactor, with glass wool plugs on both sides. Before the reaction, the catalyst was reduced in situ in flowing hydrogen (160 mL min⁻¹) using the following temperature regime: room temperature to 723 K at 1 K min⁻¹ and then held at 723 K for 2 h. After the reduction was complete, the furnace was cooled to the desired reaction temperature and the reactor system was pressurized to 6.21 MPa using a back-pressure regulator. The liquid feed (20 wt % sorbitol or 15 wt % mannitol aqueous solution, unless otherwise noted) was then co-fed to the reactor with hydrogen (40 mL min⁻¹) using an Eldex Laboratories (Napa, Calif.) 1SM HPLC pump. A 150-mL stainless-steel gas-liquid separator was used to accumulate liquid products at the top of the reactor. The gaseous products continued to flow to the back-pressure regulator to maintain the reaction pressure. The reactor was in a plexiglass enclosure for safety purposes. Both an excess flow shut-off (Parker Hannifin Corporation, Cleveland, Ohio, FS190) and a pressure relief (Swagelok®, SS-4R3A) (Swagelok, Inc., Cleveland, Ohio) valve were also installed in the reactor system to ensure safe operation. Online gas chromatography (GC; Agilent, 7890A) was used to analyze the reactor effluent gas. Hydrogen, methane, CO, and CO₂ in the gaseous products were analyzed using a thermal conductivity detector with a GS-CarbonPLOT capillary column (Agilent, Catalog No. 113-3133) (Agilent, Inc., Santa Clara, Calif.). The injection port and the detector were held at 473 K and 433 K, respectively. The column flow rate was 2 mL min⁻¹ with He carrier gas. Alkanes in the gaseous products were analyzed using a flame ionization detector with an Rt®-Q-Bond (Restek, State College, Pa., Catalog No. 19744) capillary column. The injection port and the detector were held at 473 K and 433 K, respectively. The column flow rate was 2 mL min⁻¹ with He carrier gas. The following GC oven temperature regime was used: the temperature was held at 308 K for 5 min, ramped to 423 K at 5 K min⁻¹ and kept at 423 K for 15 min. The liquid product accumulated in the gas-liquid separator was drained periodically into a collecting container in which cyclohexane (Acros Organics, 99+%) was preloaded, and passed through a 0.45-μm membrane filter. The aqueous phase and organic phase were analyzed using GC-MS (Shimadzu, GC-2010) and offline GC (Agilent, 7890A) with a flame ionization detector and an Rtx®-VMS (Restek, Catalog No. 19915) capillary column. For each analysis, 1 μL of liquid sample was injected. For GC-MS, both the injection port and the detector were held at 513 K. The column flow rate was 1.57 mL min⁻¹ with He carrier gas. The following GC oven temperature regime was used: the temperature was held at 308 K for 5 min, ramped to 323 K at 5 K min⁻¹, then to 513 K at 20 K min⁻¹, and kept at 513 K for 7.5 min. For offline GC, both the injection port and the detector were held at 513 K. The column flow rate was 1.24 mL min⁻¹ with He carrier gas. The following GC oven temperature regime was used: the temperature was held at 313 K for 5 min, ramped to 513 K at 7.5 K min⁻¹, and kept at 513 K for 15 min. The aqueous phase was analyzed using high-performance liquid chromatography (HPLC; Shimadzu© LC-20AT) with ultraviolet-visible (UV-Vis; SPD-20AV) and refractive index (RID-10A) detectors. A Biorad® Aminex HPX-87H column (Biorad, Inc., Hercules, Calif.; Catalog No. 125-0140) was used at 303 K with 0.005 M H₂SO₄ as the mobile phase, flowing at a rate of 0.6 mL min⁻¹. For each analysis, 1 μL of liquid sample was injected into the HPLC.

Conversion, molar carbon selectivity, and yield were calculated according to the following equations:

$$Conversion ( \% ) = \frac{{C}_{feed , inlet} {V}_{inlet} - {C}_{feed , aqueous} {V}_{aqueous}}{{C}_{feed , inlet} {V}_{inlet}} \times 100$$

$$Molar carbon selectivity ( \% ) = \frac{\begin{matrix}{C}_{product , aqueous} {V}_{aqueous} + {C}_{product , organic} {V}_{organic} + \\ {C}_{product , gaseous} {V}_{gaseous}\end{matrix}}{{C}_{feed , inlet} {V}_{inlet} - {C}_{feed , aqueous} {V}_{aqueous}} \times 100$$

$$Yield ( \% ) = \frac{\begin{matrix}{C}_{product , aqueous} {V}_{aqueous} + {C}_{product , organic} {V}_{organic} + \\ {C}_{product , gaseous} {V}_{gaseous}\end{matrix}}{{C}_{feed , inlet} {V}_{inlet}} \times 100$$

where C_{feed,inlet} and C_{feed,aqueous} are the concentration of carbon in the feedstock at the input and output, respectively; C_{product} is the concentration of carbon in the product at the corresponding phase; V is the corresponding phase volume. Product carbon selectivity was defined as the ratio of carbon moles of product divided by the total carbon moles of corresponding categories (light gases, gasoline-range products, and aqueous-phase products).

Before conducting measurements with different space velocities, the catalysts had been running at a contact time (W/Fₕₑₓᵢₜₒₗₛ) of 5.8 g_{cat.}·s·mmol⁻¹ at 518 K for 70 h. At least three liquid samples were collected for each reaction condition at regular intervals after steady-state performance had been attained with time-on-stream in the gas phase. No measurable deactivation was detected during this period over both the Pt/Zr—P and Pt—ReOₓ/C catalysts.

### Results:

Table 1 shows selected data for the production of α,ω-diols over an iridium-rhenium catalyst on a carbon support (Ir—Re/C catalyst). Sorbitol was used a model biomass-derived compound to produce α,ω-diol precursors (e.g., triols and cyclic ethers). Temperatures ranged from 393 K to 518 K. Reactions were run at 250, 500, and 900 psig. The yield of α,ω-diol precursors was significantly increased by changing space velocity, reaction temperature, reaction pressure. Ir—Re/C gave α,ω-diol precursors with high selectivity at 518 K.

The reaction scheme at the bottom of Table 1 shows the progression of chemical transformations from the starting complex biomass to the ultimate diol product.

TABLE 1
Comparison of the catalytic performances for the hydrodeoxygenation over 5% Ir-4.8% Re/C catalyst.

| | | | | | Diol precursor selectivity/% | | | | |
|---|---|---|---|---|---|---|---|---|---|
| | | | | | Total | | α,ω-diols | Diol selectivity/% | |
| | WHSV/ | Temp./ | P/ | Conversion/ | 1,2,n- | Cyclic | precursor | | α,ω-diols |
| Feedstock | h⁻¹ | K | psig | % | triols | ethers | (C5, C6) | Total | (C5, C6) |
| 20 wt % sorbitol in water | 0.16 | 433 | 900 | 5.0 | 35.5 | 0.9 | 18.0 | 13.1 | 1.2 |
| | 0.16 | 453 | 900 | 40.0 | 36.3 | 1.1 | 13.2 | 10.8 | 1.0 |
| | 0.16 | 473 | 900 | 70.0 | 0 | 3.0 | 0 | 1.1 | 0 |
| | 0.16 | 518 | 900 | 100 | 0 | 0.5 | 0 | 0 | 0 |
| | 1.27 | 518 | 900 | 85.0 | 0 | 14.0 | 0 | 0.9 | 0 |
| | 2.92 | 518 | 900 | 74.0 | 34.1 | 7.8 | 18.1 | 26.2 | 1.4 |
| | 12.2 | 518 | 900 | 45.0 | 54.5 | 6.5 | 27.6 | 22.4 | 1.1 |
| | 12.2 | 518 | 500 | 31.0 | 55.2 | 6.5 | 27.6 | 24.0 | 1.8 |
| | 12.2 | 518 | 250 | 21.0 | 41.2 | 15.5 | 28.7 | 17.6 | 2.0 |
| 2 wt % isosorbide in | 0.16 | 393 | 250 | 6.6 | 35.8 | 0 | 20.9 | 23.3 | 13.9 |
| water | | | | | | | | | |
| 1,2,6-hexanetriol in water | 0.16 | 393 | 250 | 11.2 | — | 6.1 | 6.1 | 81.4 | 70.5 |
| 5 wt % tetrahydrofurfuryl | 0.16 | 393 | 250 | 44.3 | 0 | 0.2 | 0 | 99.8 | 98.9 |
| alcohol in water | 0.52 | 393 | 500 | 23.7 | 0 | 2.3 | 0 | 92.4 | 92.1 |

[chemical structure]

Additionally, the subject method can be used to produce for 1,5-pentandiol using tetrahydrofurfuryl alcohol as the model, biomass-derived α,ω-diol precursor. The Ir—Re/C catalyst selectively produced 1,5-pentanediol at selectivity of 98.9% at 393 K. Additional testing was also performed using sorbitol as the feed, and two different catalysts: 4% platinum on phosphate-bound zirconia (4% Pt/Zr—P) and 4% platinum and 4% rhenium on phosphate-bound zirconia (4% Pt-4% ReOₓ/Zr—P). The two parallel reactions were run at the same WHSV, pressure, and H₂ flow rate. The only different parameters were the temperature and the catalyst used. Complete reaction conditions were as follows:

Reaction conditions 1: T=518 K, P=6.21 Mpa, WHSV=0.16 h⁻¹, feed=20 wt. % aqueous sorbitol, H₂ flow rate=40 ml min⁻¹, catalyst=4% Pt/Zr—P.

Reaction conditions 2: T=453 K, P=6.21 Mpa, WHSV=0.16 h⁻¹, feed=20 wt. % aqueous sorbitol, H₂ flow rate=40 ml min⁻¹, catalyst=4% Pt-4% ReOₓ/Zr—P.

The results are show in Table 2:

TABLE 2

| | Reaction Condition 1 | Reaction Condition 2 |
|---|---|---|
| | 4% Pt/Zr—P | 4% Pt-4% ReOₓ/Zr—P |
| Product | Yield (wt %) | Yield (wt %) |
| CO₂ | 9.8 | 0.7 |
| C₁-C₄ alkanes | 12.5 | 9.2 |
| C₁-C₄ alcohols | 22.0 | 26.5 |
| C₅-C₆ alkanes | 21.8 | 4.7 |
| C₅-C₆ alcohols | 10.8 | 23.4 |
| Aldehydes | 0.5 | 0.1 |
| Ketones | 2.7 | 3.2 |
| Tetrahydrofurans | 5.4 | 12.0 |
| Tetrahydropyrans | 11.8 | 7.2 |
| Polyols | 2.7 | 12.3 |
| Isosorbide, sorbitan, | n/a | 0.7 |
| mannitol | | |

As can be seen from Table 2, by adjusting the reaction conditions, and the concentration of metal to acid sites, the selectivity of the reaction can be controlled. Notably, the total yield of C5 and C6 products combined under reaction 1 conditions was 51.9 wt % and under reaction 2 conditions was 56.2%. Broken out by C5 versus C6 products, the theoretical yield for C5 α,ω-diols via these two reactions is 57.8 wt %. The theoretical yield for C6 α,ω-diols via these two reactions is C6: 65.5%.

### Catalytic Activity:

The APHDO of sorbitol was carried out at 518 K and 6.21 MPa as a function of WHSV over Pt/Zr—P and Pt—ReOₓ/C, as shown Table 3. The products included C1-C6 alkanes, C1-C6 alcohols, polyols, and larger intermediates formed during the reaction. We have grouped the products into three major categories: 1) light gases (C1-C4 alkanes, CO, and CO₂), 2) gasoline-range products (C5-C6 alkanes, and C2-C6 monofunctional compounds such as alcohols, aldehydes, cyclic ethers, carboxylic acids and ketones), and 3) aqueous-phase oxygenates (methanol, polyols, mannitol, sorbitan, and isosorbide) (N. Li, G. A. Tompsett, G. W. Huber, ChemSusChem 3 (2010) 1154-1157. N. Li, G. W. Huber, Journal of Catalysis. 270 (2010) 48-59. N. Li, G. A. Tompsett, T. Zhang, J. Shi, C. E. Wyman, G. W. Huber, Green Chemistry 13 (2011) 91-101.) The product yields are strongly related to the WHSV. The sorbitol conversion and the yield of light gases increased with decreasing space velocity for both catalysts.

In the case of Pt/Zr—P, the yield of gasoline-range products increased from 1.4% to 66.8% with decreasing space velocity from 2.92 to 0.16 h⁻¹. The yield of aqueous-phase products went through a maximum with space velocity where the maximum aqueous phase product yield was obtained at 0.73 h⁻¹ with this same catalyst. Most of the carbon was in the liquid phase. The yield of light gases was less than 19.1%. The yield of gasoline-range products increased from 3.2% to 44.4% with decreasing space velocity from 46.5 to 2.92 h⁻¹ for the Pt—ReOₓ/C catalyst. The yield of gasoline-range products then decreased as the space velocity further decreased to below 2.91 h⁻¹. The yield of the aqueous-phase products went through a maximum at 12.2 h⁻¹ for the Pt—ReOₓ/C catalyst. At a WHSV of 0.16 h⁻¹ most of the products (83.5% yield) were light gases for this same catalyst.

Pt—ReOₓ/C was 1 to 9 times more active than Pt/Zr—P on a mass basis. Even at a WHSV of 46.5 h⁻¹, 29.7% of sorbitol was converted for the Pt—ReOₓ/C catalyst. In contrast, for Pt/Zr—P the sorbitol conversion was 10.9% at a WHSV of 2.92 h⁻¹. Pt/Zr—P gave a higher yield (66.8%) of gasoline-range products than Pt—ReOₓ/C (44.4%) at 518 K. The RONs of the catalyst for the gasoline-range products were 80.0 and 89.5, respectively, which is comparable to commercial gasoline, which has a RON of at least 87 (Table 3).

The product distribution at 518 K and 6.21 MPa in both the gas and liquid phases over Pt/Zr—P and Pt—ReOₓ/C are listed in Table 3. The product selectivities were calculated on a carbon basis. At high space velocities, CO₂ was the main product in the light gases. No CO peak was detected for the Pt/Zr—P, whereas for the Pt—ReOₓ/C a trace of CO was detected with a selectivity of less than 1.0%. The CO to CO₂ ratios obtained over the Pt—ReOₓ/C decreased from 0.02 to 0 as the space velocity increased. These are the expected products of decarbonylation of aldehydic species followed by water-gas shift reactions. (G. W. Huber, R. D. Cortright, J. A. Dumesic, Angewante Chemistry International. Edition 43 (2004) 1549-1551. N. Li, G. W. Huber, J. Catal. 270 (2010) 48-59.) The C1-C4 alkane selectivity generally increased with decreasing space velocity, whereas the CO₂ selectivity decreased for both catalysts. The C1-C4 alkane distribution shifted to heavier alkanes for Pt/Zr—P as the space velocity decreased from 2.92 to 0.16 h⁻¹. Similar patterns were also observed for Pt—ReOₓ/C as the space velocity decreased from 46.5 to 2.92 h⁻¹. As the space velocity decreased further, the C1-C4 alkane distribution shifted to lighter alkanes for Pt—ReOₓ/C. This shift implies the occurrence of C—C bond-cleaving reactions. It is noticeable that about 19.1% of the carbon in the light gases was methane at a space velocity of 0.16 h⁻¹ over Pt—ReOₓ/C. This suggests that the Pt—ReOₓ/C might be able to hydrogenate the CO and CO₂ into methane.

TABLE 3
Total molar carbon selectivity of products in gas- and liquid-phase as a function of WHSV
over Pt/Zr-P and Pt-ReQₓ/C for aqueous phase hydrodeoxygenation of 20 wt % sorbitol^{a}

| Catalysts | Pt/Zr-P | | | Pt-ReOₓ/C | | | | | |
|---|---|---|---|---|---|---|---|---|---|
| WHSV (h⁻¹) | 0.16 | 0.73 | 2.92 | 0.16 | 1.27 | 2.92 | 12.2 | 20.8 | 46.5 |
| Conversion (%) | 99.5 | 43.4 | 10.9 | 100 | 100 | 98.9 | 70.2 | 62.9 | 29.7 |
| Light gas yield (%) | 19.1 | 3.8 | 1.2 | 83.5 | 63.8 | 37.8 | 8.8 | 5.8 | 2.0 |
| C1-C4 light gas carbon selectivity (%) | | | | | | | | | |
| CO₂ | 43.9 | 95.3 | 95.0 | 47.7 | 60.0 | 66.5 | 77.9 | 78.3 | 78.7 |
| Methane | 5.9 | 1.1 | 1.8 | 19.1 | 8.9 | 5.3 | 3.3 | 3.4 | 3.0 |
| Ethane | 16.4 | 2.3 | 2.5 | 13.7 | 10.8 | 7.8 | 4.7 | 4.7 | 4.5 |
| Propane | 16.6 | 0.9 | 0.6 | 10.9 | 11.1 | 9.9 | 5.3 | 5.2 | 4.8 |
| Butane | 17.2 | 0.4 | 0.1 | 8.5 | 9.2 | 10.5 | 8.7 | 8.4 | 8.5 |
| Gasoline range product yield (%) | 66.8 | 7.4 | 1.4 | 14.7 | 40.1 | 44.4 | 15.2 | 13.4 | 3.2 |
| Gasoline products carbon selectivity (%) | | | | | | | | | |
| Pentane | 8.1 | 0.4 | 0.6 | 34.3 | 9.1 | 6.6 | 5.0 | 3.8 | 4.4 |
| Hexane | 19.8 | 0.2 | 0.3 | 23.4 | 8.4 | 9.2 | 8.4 | 7.4 | 9.7 |
| Ethanol | 6.9 | 17.1 | 40.6 | 2.4 | 9.3 | 11.6 | 7.4 | 5.2 | 6.4 |
| Propanol | 16.6 | 6.5 | 7.2 | 29.8 | 13.4 | 12.4 | 6.6 | 4.8 | 4.5 |
| Propionic acid | 2.2 | 0 | 0 | 0 | 0.3 | 0.2 | 0.1 | 0.2 | 0 |
| Acetone | 0 | 1.1 | 0 | 1.2 | 0.7 | 0.5 | 0.2 | 0.1 | 0 |
| Butanol | 4.2 | 1.2 | 1.1 | 7.8 | 6.8 | 5.9 | 2.0 | 1.6 | 0.9 |
| Butanone | 0 | 0.6 | 1.2 | 0.1 | 0.7 | 0.3 | 0.2 | 0.2 | 0.1 |
| Butanal | 0.4 | 0.9 | 0 | 0 | 0 | 0 | 0 | 0.3 | 1.9 |
| Tetrahydrofuran | 1.3 | 0.2 | 0 | 0 | 2.6 | 2.6 | 0.9 | 0.9 | 0.3 |
| Hydroxy-tetrahydrofuran | 0 | 0 | 0 | 0 | 0 | 0.3 | 0.4 | 0.2 | 0 |
| Butanoic acid | 1.6 | 7.5 | 7.8 | 0 | 0.2 | 0.2 | 1.1 | 0.6 | 0.2 |
| Pentanol | 4.2 | 0.7 | 0 | 0.3 | 8.4 | 4.0 | 1.8 | 1.5 | 1.4 |
| Pentanone | 2.4 | 0.3 | 0 | 0 | 8.7 | 5.7 | 3.0 | 3.3 | 1.8 |
| Tetrahydropyran | 2.7 | 0 | 0 | 0.1 | 4.3 | 4.2 | 1.4 | 1.8 | 0.4 |
| Tetrahydro-4H-pyran-4-ol^{b} | 0.1 | 2.1 | 1.1 | 0 | 1.2 | 2.2 | 4.1 | 4.6 | 4.5 |
| Methyl-tetrahydrofuran | 1.2 | 0.5 | 0 | 0 | 4.1 | 3.9 | 2.3 | 2.9 | 1.1 |
| Tetrahydro-furfuryl alcohol | 0.9 | 14.4 | 12.0 | 0 | 0 | 2.2 | 4.9 | 5.2 | 6.0 |
| Pentanoic acid | 0.1 | 0 | 0 | 0 | 0 | 2.0 | 2.4 | 2.4 | 0 |
| Hexanol | 9.8 | 12.3 | 1.5 | 0 | 10.0 | 7.5 | 9.0 | 10.0 | 9.4 |
| Hexanone | 1.0 | 0.8 | 0 | 0 | 1.2 | 0.8 | 0.7 | 5.1 | 5.1 |
| Dimethyl-tetrahydrofuran | 3.1 | 7.0 | 13.6 | 0.7 | 4.2 | 4.4 | 6.5 | 7.8 | 8.4 |
| Methyltetrahydrofuran-methanol^{b} | 0.3 | 19.1 | 0 | 0 | 0.3 | 3.6 | 7.1 | 8.0 | 8.7 |
| Tetrahydro-methoxymethyl-furan^{b} | 0.2 | 11.9 | 0 | 0 | 0.2 | 2.0 | 3.7 | 3.2 | 10.4 |
| Methyl-tetrahydropyran | 9.3 | 3.0 | 0 | 0 | 5.8 | 5.1 | 13.6 | 11.3 | 6.6 |
| Tetrahydropyran-methanol | 3.1 | 2.0 | 3.8 | 0 | 0 | 1.5 | 3.8 | 3.3 | 3.3 |
| Hexanoic acid | 0.6 | 2.2 | 9.3 | 0 | 0.2 | 1.0 | 3.7 | 3.5 | 4.5 |
| Estimated research octane number (RON)^{c} | 80.0 | 99.2 | 110.4 | 78.3 | 86.2 | 89.5 | 87.0 | 84.5 | 83.1 |
| Aqueous phase product yield (%) | 2.8 | 27.5 | 7.3 | 7.5 | 1.0 | 18.8 | 53.3 | 49.3 | 27.2 |
| Aqueous cut carbon selectivity (%) | | | | | | | | | |
| Methanol | 12.7 | 0.7 | 1.3 | 100 | 76.4 | 4.8 | 0.5 | 0.4 | 0.7 |
| Ethylene glycol | 0 | 17.7 | 19.4 | 0 | 0 | 8.3 | 5.3 | 2.8 | 4.3 |
| 1,2-Propanediol | 0.5 | 27.6 | 28.2 | 0 | 0 | 15.7 | 5.4 | 5.8 | 3.8 |
| 1,3-Propanediol | 0 | 0 | 0 | 0 | 0 | 0 | 0 | 0 | 0 |
| Glycerol | 6.3 | 5.3 | 12.4 | 0 | 0 | 0 | 14.1 | 15.0 | 15.0 |
| Hydroxyacetone | 0 | 0.4 | 2.0 | 0 | 0 | 0.2 | 0.2 | 0.1 | 0.5 |
| 1,2-Butanediol | 6.6 | 6.9 | 2.7 | 0 | 1.6 | 4.9 | 2.2 | 3.1 | 1.7 |
| 1,4-Butanediol | 2.5 | 1.0 | 0 | 0 | 0 | 4.5 | 2.0 | 0.8 | 0.6 |
| 2,3-Butanediol | 20.1 | 4.9 | 0.7 | 0 | 16.9 | 2.9 | 0.7 | 0.9 | 0 |
| 1,2-Pentanediol | 4.3 | 1.5 | 0 | 0 | 5.1 | 4.0 | 2.3 | 1.2 | 1.0 |
| 1,4-Pentanediol | 8.0 | 1.0 | 0.2 | 0 | 0 | 2.2 | 0.8 | 1.0 | 0.3 |
| 1,5-Pentanediol | 11.2 | 1.6 | 0 | 0 | 0 | 1.6 | 1.0 | 1.1 | 0 |
| 1,2-Cyclopentanediol | 0 | 0 | 0 | 0 | 0 | 0 | 0 | 0 | 0 |
| 1,2-Hexanediol | 3.1 | 2.6 | 0 | 0 | 0 | 4.8 | 0 | 0.5 | 0.3 |
| 1,6-Hexanediol | 6.1 | 1.7 | 0.2 | 0 | 0 | 1.2 | 0.5 | 0.5 | 0.3 |
| 2,5-Hexanediol | 14.7 | 0.9 | 0.2 | 0 | 0 | 0.6 | 0.1 | 0.1 | 0.3 |
| 1,2-Cyclohexanediol | 0 | 0 | 0 | 0 | 0 | 0.2 | 0 | 0.3 | 0 |
| 1,2,6-Hexanetriol | 0 | 5.0 | 4.7 | 0 | 0 | 0 | 4.0 | 4.9 | 8.8 |
| Mannitol | 0 | 0 | 0 | 0 | 0 | 0 | 22.9 | 24.7 | 47.4 |
| Sorbitan | 3.8 | 7.6 | 2.0 | 0 | 0 | 24.3 | 23.6 | 22.0 | 5.0 |
| Isosorbide | 0 | 16.4 | 26.1 | 0 | 0 | 19.8 | 14.4 | 15.1 | 10.1 |
| Carbon identified (%) | 89.2 | 95.4 | 98.9 | 106.0 | 105.2 | 103.8 | 107.1 | 98.5 | 102.8 |
| Hydrogen consumption (mol_{H2}mol_{sorbitol}⁻¹) | 3.95 | 0.65 | 0.11 | 4.62 | 3.71 | 3.07 | 1.21 | 0.97 | 0.38 |

^{a}Reaction condition: 518K, 6.21 MPa with 20 wt % sorbitol solution as a feed, and flow rate of H₂ is 40 ml min⁻¹.
^{b}Tetrahydro-4H-pyran-4-ol (85%), 5-Methyltetrahydrofuran-2-methanol (88%), and Tetrahydro-2 methoxymethyl-furan (89%) is identified by GC/MS.
^{c}Research octane number (RON) of the gasoline range products were estimated according to the method introduced in the literature, N. Li, G.A. Tompsett, T. Zhang, J. Shi, C.E. Wyman, G.W. Huber, Green Chemistry 13 (2011) 91-101..

Referring now to FIGS. 5A and 5B, these two drawings are graphs depicting the catalytic performance for the reaction of furfuryl alcohol over 10% Co/γ-Al₂O₃ catalyst, T=413 K, P=2.1 Mpa, WHSV=0.6 h⁻¹, feedstock=2 wt % furfuryl alcohol aqueous solution; H₂ flow rate=40 ml

min⁻¹. FIG. 5A depicts conversion percentage to tetrahydrofurfuryl alcohol (THFA) as a function of time on stream. FIG. 5B depicts the carbon selectively percentage to THFA (solid circles) and ratio of 1,5-propanediol to 1,2-pentanediol (open circles) as function of time on stream. The significance of FIGS. 5A and 5B are that they clearly demonstrate that the method can be configured to drive the result product mixture to contain a far greater percentage of desirable, value-added chemicals such as 1,5-pentanediol. The selective formation of α,ω-diols from cyclic ethers such as furfuryl alcohol is favored at low temperatures because the reaction is exothermic and thermodynamically limited. As shown in FIG. 5B, the Co/γ-Al₂O₃ catalyst produced 37.6% of diols from furfuryl alcohol at 413 K. This catalyst selectively produced 1,5-pentanediol at a diol selectivity of between 57.8 and 67.6%.

As noted above, the catalysts were evaluated for BET surface area, pore volume, and average pore diameter. Table 4 lists the physicochemical properties of the Pt/Zr—P and Pt—ReOₓ/C catalysts before reaction. Pt/Zr—P had a two times lower BET surface area and 1.8 times lower pore volume than the support itself. The noticeable decrease in surface area after the Pt addition could be attributed to the pore blockage by aggregates of Pt clusters. In contrast, even with high metal loading, little decrease in BET surface area and pore volume was obtained when Pt and Re were added to a carbon support.

TABLE 4
Physisochemical properties of Pt/Zr-P and Pt-ReOₓ/C before and after reaction.

| | | | | H₂ chemisorption | | | | | | |
|---|---|---|---|---|---|---|---|---|---|---|
| | | Total pore | Average pore | at 298 K | | Acid amount | | | Acid/Metal | Carbon |
| | S_{BET} | volume | diameter^{a} | H₂ uptake | H/Pt | Total^{b} | n_{Brønsted}^{c} | Brøsted/Lewis | ratio | deposit^{d} |
| Catalysts | (m²/g) | (cm³/g) | (nm) | (μmol H₂/g) | (%) | (μmol/g) | (μmol/g) | ratio | (NH₃/H) | (wt. %) |
| Pt/Zr-P | 178 | 0.58 | 3.71 | 15.1 | 14.7 | 2316.3 | 548.1 | 0.31 | 76.7 | — |
| Pt/Zr-P after the | 5 | 0.10 | 3.13 | 2.1 | 2.0 | 110.0 | 15.2 | 0.16 | 26.2 | 15.9 |
| reaction for 114 h^{e} | (12) | (0.08) | (4.42) | | | | | | | |
| Pt/Zr-P after the | 7 | 0.04 | 3.49 | 1.1 | 1.0 | 109.6 | 16.7 | 0.18 | 49.8 | 1.2 |
| reaction for 121 h^{f} | (8) | (0.07) | (2.00) | | | | | | | |
| Pt-ReOₓ/C | 621 | 0.84 | 3.94 | 33.5 | 25.6 | 167.5 | 36.6 | 0.28 | 2.5 | — |
| Pt-ReOₓ/C after the | 518 | 0.69 | 3.93 | 122.2 | 93.5 | 97.8 | 23.1 | 0.31 | 0.4 | 6.8 |
| reaction for 140 h^{e} | | | | | | | | | | |

^{a}The average pore diameter was determined by the BJH desorption method.
^{b}The total acidity was determined by quantifying the desorbed NH₃ by NH₃-TPD.
^{c}The amount of Brønsted acid sites was determined by quantifying the desorbed propylene by IPA-TPD.
^{d}The amount of carbon on the used catalysts were determined with TOC analyzer.
^{e}Reaction condition: 518K, 6.21 MPa with 20 wt % sorbitol solution as a feed, and flow rate of H₂ is 40 ml min⁻¹. The data in parenthesis correspond the N₂ physisorption results obtained the used catalyst after calcination at 723K for 2 h.
^{f}Reaction condition: 518K, 6.21 MPa with 15 wt % mannitol solution as a feed, and flow rate of H₂ is 40 ml min⁻¹. The data in parenthesis correspond the N₂ physisorption results obtained the used catalyst after calcination at 723K for 2 h.

What is claimed is:

1. A method to make triols and diols, the method comprising:
performing an hydrodeoxygenation reaction on a feedstock comprising:
(i) a biomass-derived reactant, and
(ii) hydrogen,
by contacting the feedstock with a heterogeneous metal-comprising bifunctional catalyst or a combination of two or more heterogeneous metal-comprising catalysts that catalyze cleavage of C—C and C—O bonds, for a temperature, pressure, and weight hourly space velocity to yield a product mix comprising triols, diols, or combinations thereof.

2. The method of claim 1, wherein the feedstock is contacted with the catalyst or catalysts for a temperature, pressure, and weight hourly space velocity to yield a product mix comprising α,ω-diols.

3. The method of claim 2, wherein the hydrodeoxygenation reaction is carried out at a temperature from about 100° C. to about 500° C., a pressure from about 100 psig to about 1000 psig, a weight-hourly space velocity from about 0.1 to about 50.0 gram of liquid feedstock per gram of catalyst per hour, and hydrogen is present in stoichimetric excess for reaction of the biomass-derived reactant to a diol product.

4. The method of claim 2, wherein the feedstock is contacted with the catalyst or catalysts for a time, temperature, pressure, and weight hourly space velocity to yield a product mix comprising C₂ to C₁₂-α,ω-diols.

5. The method of claim 2, wherein the feedstock is contacted with the catalyst or catalysts for a time, temperature, pressure, and weight hourly space velocity to yield a product mix comprising C₂ to C₆-α,ω-diols.

6. The method of claim 1, wherein the catalyst or catalysts are adhered to a support.

7. The method of claim 6, wherein the support comprises a solid acid having Lewis acid functionality or Bronsted acid functionality.

8. The method of claim 6, wherein the support is selected from the group consisting of carbon, a refractory oxide, a phosphate-bonded refractory oxide, and combinations thereof.

9. The method of claim 8, wherein the feedstock is contacted with the catalyst or catalysts for a time, temperature, pressure, and weight hourly space velocity to yield a product mix comprising α,ω-diols.

10. The method of claim 9, wherein the feedstock is contacted with the catalyst or catalysts for a time, temperature, pressure, and weight hourly space velocity to yield a product mix comprising C₂ to C₁₂-α,ω-diols.

11. The method of claim 9, wherein the feedstock is contacted with the catalyst or catalysts for a time, temperature, pressure, and weight hourly space velocity to yield a product mix comprising C₂ to C₆-α,ω-diols.

12. The method of claim 6, wherein the catalyst or catalysts comprises a metal selected from the group consisting of Pt, Pd, Ru, Ir, W, Co, Ni, Fe, alloys thereof, mixtures thereof, and combinations thereof.

13. The method of claim 12, wherein catalyst or catalysts are further combined with or alloyed with one or more additional metals selected from the group consisting of alkali metals, alkaline earth metals, and rare earth metals.

14. The method of claim 12, wherein the feedstock is contacted with the catalyst or catalysts for a time, temperature, pressure, and weight hourly space velocity to yield a product mix comprising α,ω-diols.

15. The method of claim 14, wherein the feedstock is contacted with the catalyst or catalysts for a time, temperature, pressure, and weight hourly space velocity to yield a product mix comprising C₂ to C₁₂-α,ω-diols.

16. The method of claim 14, wherein the feedstock is contacted with the catalyst or catalysts for a time, temperature, pressure, and weight hourly space velocity to yield a product mix comprising C₂ to C₆-α,ω-diols.

17. The method of claim 1, comprising performing an aqueous-phase hydrodeoxygenation reaction on a feedstock comprising a biomass-derived reactant in aqueous solution, wherein hydrogen is present in about 10× to 1,000× stoichimetric excess for reaction of the biomass-derived reactant to a diol product.

18. A method to make triols and diols, the method comprising:
performing an aqueous-phase hydrodeoxygenation reaction on a feedstock comprising:
(i) a biomass-derived reactant in aqueous solution, and
(ii) hydrogen in stoichimetric excess for reaction of the biomass-derived reactant to a diol product,
by contacting the feedstock with a heterogeneous metal-comprising bifunctional catalyst or a combination of two or more heterogeneous metal-comprising catalysts that catalyze cleavage of C—C and C—O bonds,
at a temperature from about 100° C. to about 500° C., a pressure from about 100 psig to about 1000 psig, and a weight-hourly space velocity from about 0.1 to about 50.0 gram of liquid feedstock per gram of catalyst per hour,
wherein the catalyst or catalysts comprise a metal selected from the group consisting of Pt, Pd, Ru, Ir, W, Co, Ni, Fe, alloys thereof, mixtures thereof, and combinations thereof,
wherein the catalyst or catalysts are adhered to a support selected from the group consisting of carbon, a refractory oxide, a phosphate-bonded refractory oxide, and combinations thereof; and
wherein the reaction yields a product mix comprising triols, diols, or combinations thereof.

19. The method of claim 18, wherein catalyst or catalysts are further combined with or alloyed with one or more additional metals selected from the group consisting of alkali metals, alkaline earth metals, and rare earth metals.

20. The method of claim 18, wherein the feedstock is contacted with the catalyst or catalysts for a time, temperature, pressure, and weight hourly space velocity to yield a product mix comprising α,ω-diols.
